(19) United States
(12) Patent Application Publication
(10) Pub. No.: US 2026/0341520 A1
(43) Pub. Date: Oct. 29, 2026

(54) Lifestyle Informed Personalized Blood Tests Ranges and Risk Assessment

(71) Applicant: Google LLC, Mountain View, CA (US)

(72) Inventors: Naghmeh Rezaei, Redwood City, CA (US); Javier L. Prieto, Oakland, CA (US); Abbas-Ali Heydari, Tracy, CA (US)

(21) Appl. No.: 19/111,681

(22) PCT Filed: Sep. 14, 2023
(86) PCT No.: PCT/US2023/032721

§ 371 Date: Mar. 13, 2025

Related U.S. Application Data
(60) Provisional application No. 63/406,624, filed on Sep. 14, 2022.

(51) Int. Cl. G16H 50/30; G16H 50/20

(52) U.S. Cl. CPC G16H 50/30; G16H 50/20

(57) ABSTRACT

Systems, devices, computer-implemented methods, and non-transitory computer-readable media that facilitate health-based predictions for a patient using a single time point biological sample of the patient are provided. A regularization component can be integrated into a triplet-based objective function associated with a triplet-based machine learning model to create a modified triplet-based objective function. The regularization component can operate to regularize distances between pairs of positive data values and negative data values in triplet tuples of a dataset with respect to anchor values in the triplet tuples. A triplet-based machine learning model can be trained using the dataset and the modified triplet-based objective function to create a trained triplet-based machine learning model. The trained triplet-based machine learning model can be implemented to output one or more health-based predictions for a patient based at least in part on a single time point biological sample of the patient.

### PRIORITY CLAIM

The present application claims priority to U.S. Provisional Patent Application No. 63/406,624 having a filing date of Sep. 14, 2022. Applicant claims priority to and the benefit of said application and incorporates said application herein by reference in its entirety.

### FIELD

The present disclosure relates generally to health risk modeling. More particularly, the present disclosure relates to health risk modeling using a single time point biological sample of a patient.

### BACKGROUND

Predicting patients' potential health risks remains a major area of focus and a challenge in healthcare. Standardized lab tests are widely used for health evaluation of patients, differential diagnosis, and treatment. A problem with interpretation of these data is the lack of quantitative and personalized metrics. Most of the current standardized reference values are established based on percentiles of different age and gender groups. However, age and gender alone can explain less than 10 percent (%) of the within-normal test variance in most tests. Additionally, personalized models based on patients' history can explain 60% of the variance for half of the tests. These difficulties are further exacerbated if the task is to assess future health risks using only a single time point of blood measurements. In fact, not only do most risk prediction models rely on multiple measurements, the definition of health risks are also based on multiple time points.

### SUMMARY

Aspects and advantages of embodiments of the present disclosure will be set forth in part in the following description, or can be learned from the description, the appended claims, and/or the accompanying drawings, or can be learned through practice of the embodiments.

According to an example embodiment, a computing system can include one or more processors. The computing system can further include one or more non-transitory computer-readable media that collectively store instructions that, when executed by the one or more processors, cause the computing system to perform operations. The operations can include training a triplet-based machine learning model using a dataset and a modified triplet-based objective function to create a trained triplet-based machine learning model. The dataset can include a plurality of data values that can be represented graphically in a multi-dimensional space. For instance, the dataset can be a healthcare dataset having at least one of genetic features, phenotypic features, or lifestyle features. The operations can further include receiving a single time point biological sample of a patient, and generating one or more health-based predictions for the patient using the trained triplet-based machine learning model and the single time point biological sample of the patient. The modified triplet-based objective function can include a regularization component that can operate to regularize distances between pairs of positive data values and negative data values in triplet tuples of the dataset with respect to anchor values in the triplet tuples.

According to another example embodiment, a computer-implemented method can include integrating, by a computing system that can include one or more computing devices, a regularization component into a triplet-based objective function associated with a triplet-based machine learning model to create a modified triplet-based objective function. The regularization component can operate to regularize distances between pairs of positive data values and negative data values in triplet tuples of a dataset with respect to anchor values in the triplet tuples. The dataset can include a plurality of data values that can be represented graphically in a multi-dimensional space. For instance, the dataset can be a healthcare dataset having at least one of genetic features, phenotypic features, or lifestyle features. The computer-implemented method can further include training, by the computing system, a triplet-based machine learning model using the dataset and the modified triplet-based objective function to create a trained triplet-based machine learning model. The computer-implemented method can further include generating, by the computing system, one or more health-based predictions for a patient using the trained triplet-based machine learning model and a single time point biological sample of the patient.

According to another example embodiment, a computing device can include one or more processors. The computing device can further include one or more non-transitory computer-readable media that collectively store instructions that, when executed by the one or more processors, cause the computing device to perform operations. The operations can include integrating a regularization component into a triplet-based objective function associated with a triplet-based machine learning model to create a modified triplet-based objective function. The regularization component can operate to regularize distances between pairs of positive data values and negative data values in triplet tuples of a dataset with respect to anchor values in the triplet tuples. The dataset can include a plurality of data values that can be represented graphically in a multi-dimensional space. For instance, the dataset can be a healthcare dataset having at least one of genetic features, phenotypic features, or lifestyle features. The operations can further include training a triplet-based machine learning model using the dataset and the modified triplet-based objective function to create a trained triplet-based machine learning model that, when implemented, generates one or more health-based predictions for a patient based on a single time point biological sample of the patient.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 depicts a block diagram of an example, non-limiting computing system that can perform health-based predictions for a patient using a single time point biological sample of the patient according to example embodiments of the present disclosure.

FIG. 2 depicts a block diagram of an example, non-limiting computing device that can perform health-based predictions for a patient using a single time point biological sample of the patient according to example embodiments of the present disclosure.

FIG. 3 depicts a block diagram of an example, non-limiting computing device that can perform health-based predictions for a patient using a single time point biological sample of the patient according to example embodiments of the present disclosure.

FIG. 4 depicts an example, non-limiting diagram according to example embodiments of the present disclosure.

FIG. 5 depicts an example, non-limiting diagrams of training datasets and testing datasets according to example embodiments of the present disclosure.

FIG. 6 depicts a flow chart diagram of an example, non-limiting computer-implemented method to perform health-based predictions for a patient using a single time point biological sample of the patient according to example embodiments of the present disclosure.

FIG. 7 depicts a flow chart diagram of an example, non-limiting computer-implemented method to perform health-based predictions for a patient using a single time point biological sample of the patient according to example embodiments of the present disclosure.

Repeated use of reference characters and/or numerals in the present specification and/or figures is intended to represent the same or analogous features, elements, or operations of the present disclosure. Repeated description of reference characters and/or numerals that are repeated in the present specification is omitted for brevity.

### DETAILED DESCRIPTION

### Overview

Reference now will be made in detail to embodiments of the present disclosure, one or more examples of which are illustrated in the drawings. Each example is provided by way of explanation of the present disclosure, not limitation of the disclosure. In fact, it will be apparent to those skilled in the art that various modifications and variations can be made in the present disclosure without departing from the scope or spirit of the disclosure. For instance, features illustrated or described as part of one embodiment can be used with another embodiment to yield a still further embodiment. Thus, it is intended that the present disclosure covers such modifications and variations as come within the scope of the appended claims and their equivalents.

Generally, the present disclosure is directed to performing health-based predictions for a patient using a single time point biological sample of the patient. To perform such health-based predictions for a patient using a single time point biological sample of the patient, a triplet-based objective function can be modified in accordance with example embodiments described herein such that it can provide farther separation of embeddings in different classes of a dataset (e.g., a relatively complex healthcare dataset), and thus, farther separation of the different classes when compared to existing triplet-based objective functions. Further, the triplet-based objective function can be modified in accordance with example embodiments described herein such that it can provide greater density of embeddings in each of the different classes of the dataset. As described in accordance with example embodiments of the present disclosure, by providing such increased separation between embeddings in different classes of the dataset and providing such increased density of embeddings in each of the different classes, the modified triplet-based objective function described herein can thereby provide for training a triplet-based machine learning model on a relatively complex dataset (e.g., a healthcare dataset that can include at least one of genetic features, phenotypic features, or lifestyle features), while allowing such a trained model to perform accurate classification using limited data (e.g., a single time point biological sample of the patient).

In one example embodiment, a computing system and/or computing device according to example embodiments of the present disclosure can integrate a regularization component into a triplet-based objective function associated with a triplet-based machine learning model to create a modified triplet-based objective function. In this and/or another embodiment, the regularization component can operate (e.g., when implemented using the computing system and/or computing device) to regularize distances between pairs of positive data values and negative data values in triplet tuples of a dataset with respect to anchor values in the triplet tuples. For example, in this and/or another embodiment, the regularization component can operate (e.g., when implemented using the computing system and/or computing device) to regularize the distances between the pairs of the positive data values and the negative data values in the triplet tuples of the dataset with respect to the anchor values in the triplet tuples such that a first distance between a positive data value and a negative data value in a triplet tuple of the dataset is equal to or greater than a second distance between the negative data value and an anchor value in the triplet tuple.

In another example embodiment, the computing system and/or computing device can train a triplet-based machine learning model using the dataset and the modified triplet-based objective function to create a trained triplet-based machine learning model. In this and/or another embodiment, the computing system and/or computing device can generate one or more health-based predictions for a patient using the trained triplet-based machine learning model and a single time point biological sample of the patient. In one embodiment, the one or more health-based predictions can constitute and/or include a classification of the patient in, for example: one or more predefined health-based classes (e.g., a healthy lifestyle class, an unhealthy lifestyle class, a defined blood range class, a defined health risk class); a health risk score prediction (e.g., where scores can be indicative of the likelihood that the patient will contract or develop a defined disease at some time in the future); a diagnosis prediction; a prognosis prediction; and/or another health-based prediction.

In at least one embodiment, the above-described dataset can include a plurality of data values that can be represented graphically in a multi-dimensional space. In one embodiment, the dataset can constitute and/or include a healthcare dataset that can include genetic features, phenotypic features, and/or lifestyle features.

In at least one embodiment, the above-described multi-dimensional space can constitute and/or include a Euclidean space. In this and/or another embodiment, the above-described first distance and second distance, as well as each of the above-described distances between the pairs of the positive data values and the negative data values in the triplet tuples can constitute and/or include a Euclidean distance.

In at least one embodiment, the above-described triplet-based machine learning model can constitute and/or include a triplet-based deep metric learning model. In this and/or another embodiment, the above-described trained triplet-based machine learning model can constitute and/or include a trained triplet-based deep metric learning model.

In one embodiment, the above-described modified triplet-based objective function can operate (e.g., when implemented using the computing system and/or computing device) to learn similarity-based embeddings of the plurality of data values in the dataset during training of the triplet-based machine learning model using the dataset and the modified triplet-based objective function.

In at least one embodiment, the above-described modified triplet-based objective function can operate (e.g., when implemented using the computing system and/or computing device) to provide improved density of embeddings of data values of each class of data values in the dataset to provide improved classification results across different classifier models that implement the modified triplet-based objective function compared to classifier models that implement a different objective function. In one embodiment, the modified triplet-based objective function can operate (e.g., when implemented using the computing system and/or computing device) to provide improved separability between different classes of data values in the dataset to provide improved classification results across different classifier models that implement the modified triplet-based objective function compared to classifier models that implement a different objective function. In this and/or another embodiment, the modified triplet-based objective function can operate (e.g., when implemented using the computing system and/or computing device) to provide improved separability between first embeddings of first data values of a first class of data values in the dataset and second embeddings of second data values of a second class of data values in the dataset to provide improved classification results across different classifier models that implement the modified triplet-based objective function compared to classifier models that implement another objective function.

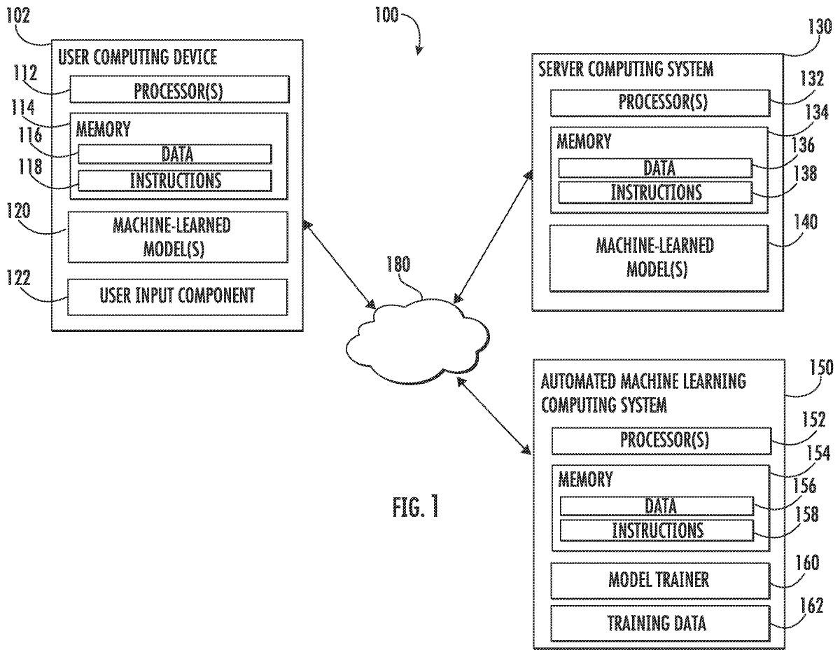

According to an example embodiment, a computing system (e.g., computing system 100, server computing system 130, and/or automated machine learning computing system 150 described below and illustrated in FIG. 1) can include one or more processors. The computing system can further include one or more non-transitory computer-readable media that collectively store instructions that, when executed by the one or more processors, cause the computing system to perform operations. The operations can include training a triplet-based machine learning model using a dataset and a modified triplet-based objective function to create a trained triplet-based machine learning model. The dataset can include a plurality of data values that can be represented graphically in a multi-dimensional space. In one embodiment, the dataset can constitute and/or include a healthcare dataset that can include genetic features, phenotypic features, and/or lifestyle features. The operations can further include receiving a single time point biological sample for a patient and generating one or more health-based predictions for the patient using the trained triplet-based machine learning model and the single time point biological sample of the patient. The modified triplet-based objective function can include a regularization component that can operate to regularize distances between pairs of positive data values and negative data values in triplet tuples of the dataset with respect to anchor values in the triplet tuples.

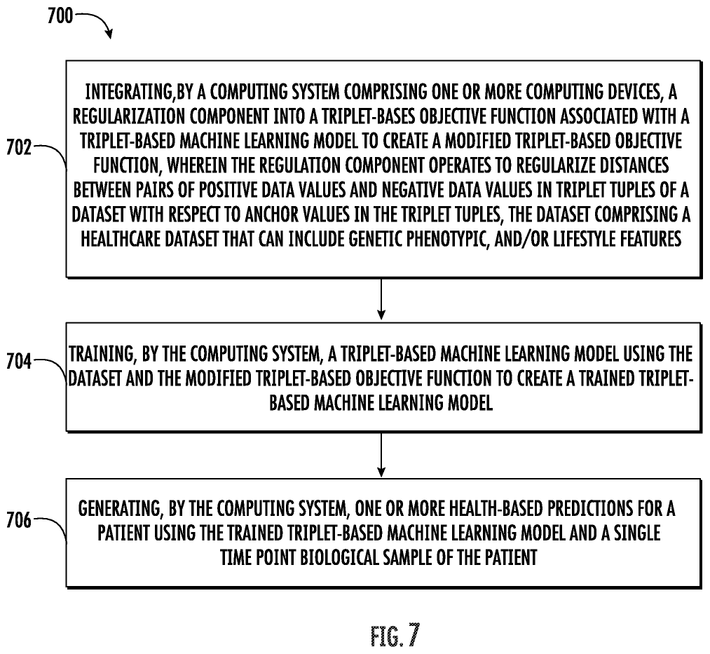

According to another example embodiment, a computer-implemented method (e.g., computer-implemented method 700 described below and illustrated in FIG. 7) can include integrating, by a computing system that can include one or more computing devices, a regularization component into a triplet-based objective function associated with a triplet-based machine learning model to create a modified triplet-based objective function. The regularization component can operate to regularize distances between pairs of positive data values and negative data values in triplet tuples of a dataset with respect to anchor values in the triplet tuples. The dataset can include a plurality of data values that can be represented graphically in a multi-dimensional space. In one embodiment, the dataset can constitute and/or include a healthcare dataset that can include genetic features, phenotypic features, and/or lifestyle features. The computer-implemented method can further include training, by the computing system, a triplet-based machine learning model using the dataset and the modified triplet-based objective function to create a trained triplet-based machine learning model. The computer-implemented method can further include receiving a single time point biological sample of a patient and generating one or more health-based predictions for the patient using the trained triplet-based machine learning model and the single time point biological sample of the patient.

According to another example embodiment, a computing device (e.g., computing device 102, computing device 200, and/or computing device 300 described below and illustrated in FIGS. 1, 2, and 3, respectively) can include one or more processors. The computing device can further include one or more non-transitory computer-readable media that collectively store instructions that, when executed by the one or more processors, cause the computing device to perform operations. The operations can include integrating a regularization component into a triplet-based objective function associated with a triplet-based machine learning model to create a modified triplet-based objective function. The regularization component can operate to regularize distances between pairs of positive data values and negative data values in triplet tuples of a dataset with respect to anchor values in the triplet tuples. The dataset can include a plurality of data values that can be represented graphically in a multi-dimensional space. In one embodiment, the dataset can constitute and/or include a healthcare dataset that can include genetic features, phenotypic features, and/or lifestyle features. The operations can further include training a triplet-based machine learning model using the dataset and the modified triplet-based objective function to create a trained triplet-based machine learning model that, when implemented, generates one or more health-based predictions for a patient based on a single time point biological sample of the patient.

The computing systems, computing devices, and/or computer-implemented methods of the present disclosure provide a number of technical effects and benefits. For instance, example embodiments of the present disclosure are directed to performing health-based predictions for a patient using a single time point biological sample of the patient. To perform such health-based predictions for a patient using a single time point biological sample of the patient, a triplet-based objective function can be modified in accordance with example embodiments described herein such that the modified version can provide farther separation of embeddings in different classes of a dataset (e.g., a relatively complex healthcare dataset), and thus, farther separation of the different classes when compared to existing triplet-based objective functions. Further, the triplet-based objective function can be modified in accordance with example embodiments described herein such that the modified version can provide greater density of embeddings in each of the different classes of the dataset when compared to existing triplet-based objective functions.

In at least one embodiment, by providing such improved separation of embeddings of different classes and such improved density of embeddings in each of the different classes of the dataset as compared to existing triplet-based objective functions, the modified version of the triplet-based objective function according to example embodiments described herein can thereby provide improved classification results when compared to existing triplet-based objective functions. Specifically, the modified version of the triplet-based objective function according to example embodiments described herein can be used to train a triplet-based machine learning model that can then be implemented to provide accurate health-based predictions for a patient using a single time point biological sample of the patient, whereas existing triplet-based machine learning models require a plurality of samples to perform the same predictions.

In at least one embodiment, by providing improved and/or accurate classification results while using less input data (e.g., a single time point biological sample of a patient), such a modified version of the triplet-based objective function according to example embodiments described herein can thereby improve processing performance (e.g., accuracy, efficiency), as well as reduce the processing workload and/or computational costs of a processor that executes a triplet-based machine learning model that has been trained using the modified version of the triplet-based objective function. In at least one other embodiment, by providing improved and/or accurate classification results while using less input data (e.g., a single time point biological sample of a patient), such a modified version of the triplet-based objective function according to example embodiments described herein can thereby improve storage capacity of a memory device that stores electronic health records of various patients that can be used by a triplet-based machine learning model that has been trained using the modified version of the triplet-based objective function.

### Example Devices and Systems

FIG. 1 depicts a block diagram of an example, non-limiting computing system 100 that can perform health-based predictions for a patient using a single time point biological sample of the patient according to example embodiments of the present disclosure. The system 100 includes a user computing device 102, a server computing system 130, and a training computing system 150 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, an FPGA, a controller, a microcontroller, etc.) and can be one processor or a plurality of processors that are operatively connected. The memory 114 can include one or more non-transitory computer-readable storage media, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

In some implementations, the user computing device 102 can store or include one or more triplet-based machine learning models 120 (e.g., trained triplet-based machine learning models and/or untrained triplet-based machine learning models 120). For example, the triplet-based machine learning models 120 can constitute and/or include one or more untrained triplet-based machine learning models and/or one or more trained triplet-based machine learning models that have been trained using the modified triplet-based objective function according to one or more embodiments described herein. In one example, the triplet-based machine learning models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Some example machine-learned models can leverage an attention mechanism such as self-attention. For example, some example machine-learned models can include multi-headed self-attention models (e.g., transformer models).

In some implementations, the one or more triplet-based machine learning models 120 can be received from the server computing system 130 over network 180, stored in the user computing device memory 114, and then used or otherwise implemented by the one or more processors 112. In some implementations, the user computing device 102 can implement multiple parallel instances of a single triplet-based machine learning model 120 (e.g., to perform parallel health-based predictions for different patients across multiple instances of single time point biological samples respectively corresponding to the different patients).

More particularly, triplet-based machine learning models 120 can be implemented in accordance with example embodiments described herein to perform health-based predictions for a patient using a single time point biological sample of the patient. To perform such health-based predictions for a patient using a single time point biological sample of the patient, a triplet-based objective function can be modified in accordance with example embodiments described herein such that it can provide farther separation of embeddings in different classes of a dataset (e.g., a relatively complex healthcare dataset), and thus, farther separation of the different classes when compared to existing triplet-based objective functions. Further, the triplet-based objective function can be modified in accordance with example embodiments described herein such that it can provide greater density of embeddings in each of the different classes of the dataset. As described in accordance with example embodiments of the present disclosure, by providing such increased separation between embeddings in different classes of the dataset and providing such increased density of embeddings in each of the different classes, the modified triplet-based objective function described herein can thereby provide for training a triplet-based machine learning model 120 on a relatively complex dataset (e.g., a healthcare dataset), while allowing such a trained triplet-based machine learning model 120 to perform accurate classification using limited data (e.g., a single time point biological sample of the patient).

Additionally or alternatively, one or more triplet-based machine learning models 140 (e.g., triplet-based machine learning models trained using the modified triplet-based objective function of the present disclosure) can be included in or otherwise stored and implemented by the server computing system 130 that communicates with the user computing device 102 according to a client-server relationship. For example, the triplet-based machine learning models 140 can be implemented by the server computing system 140 as a portion of a web service (e.g., a classification service and/or a health-based predictions service for patients). Thus, one or more triplet-based machine learning models 120 can be stored and implemented at the user computing device 102 and/or one or more models 140 can be stored and implemented at the server computing system 130.

The user computing device 102 can also include one or more user input components 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 130 includes one or more processors 132 and a memory 134. The one or more processors 132 can be any suitable processing device (e.g., a processor core, a microprocessor, an ASIC, an FPGA, a controller, a microcontroller, etc.) and can be one processor or a plurality of processors that are operatively connected. The memory 134 can include one or more non-transitory computer-readable storage media, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more triplet-based machine learning models 140. For example, the triplet-based machine learning models 140 can be or can otherwise include various machine-learned models. Example machine-learned models include neural networks or other multi-layer non-linear models. Example neural networks include feed forward neural networks, deep neural networks, recurrent neural networks, and convolutional neural networks. Some example machine-learned models can leverage an attention mechanism such as self-attention. For example, some example machine-learned models can include multi-headed self-attention models (e.g., transformer models).

The user computing device 102 and/or the server computing system 130 can train the models 120 and/or 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, an FPGA, a controller, a microcontroller, etc.) and can be one processor or a plurality of processors that are operatively connected. The memory 154 can include one or more non-transitory computer-readable storage media, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the user computing device 102 and/or the server computing system 130 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the triplet-based machine learning models 120 and/or 140 based on a set of training data 162 and/or using a modified version of a triplet-based objective function that has been modified in accordance with one or more embodiments described herein. The training data 162 can include, for example, a healthcare dataset having genetic features, phenotypic features, lifestyle features, and/or other features that can be descriptive and/or indicative of attributes and/or lifestyles of different patients.

In some implementations, if the user has provided consent, the training examples can be provided by the user computing device 102. Thus, in such implementations, the model 120 provided to the user computing device 102 can be trained by the training computing system 150 on user-specific data received from the user computing device 102. In some instances, this process can be referred to as personalizing the model.

The model trainer 160 includes computer logic utilized to provide desired functionality. The model trainer 160 can be implemented in hardware, firmware, and/or software controlling a general-purpose processor. For example, in some implementations, the model trainer 160 includes program files stored on a storage device, loaded into a memory and executed by one or more processors. In other implementations, the model trainer 160 includes one or more sets of computer-executable instructions that are stored in a tangible computer-readable storage medium such as RAM, hard disk, or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The machine-learned models described in this specification may be used in a variety of tasks, applications, and/or use cases.

In some implementations, the input to the machine-learned model(s) of the present disclosure can be text or natural language data. The machine-learned model(s) can process the text or natural language data to generate an output. As an example, the machine-learned model(s) can process the natural language data to generate a language encoding output. As another example, the machine-learned model(s) can process the text or natural language data to generate a latent text embedding output. As another example, the machine-learned model(s) can process the text or natural language data to generate a translation output. As another example, the machine-learned model(s) can process the text or natural language data to generate a classification output. As another example, the machine-learned model(s) can process the text or natural language data to generate a textual segmentation output. As another example, the machine-learned model(s) can process the text or natural language data to generate a semantic intent output. As another example, the machine-learned model(s) can process the text or natural language data to generate an upscaled text or natural language output (e.g., text or natural language data that is higher quality than the input text or natural language, etc.). As another example, the machine-learned model(s) can process the text or natural language data to generate a prediction output.

In some implementations, the input to the machine-learned model(s) of the present disclosure can be image data. The machine-learned model(s) can process the image data to generate an output. As an example, the machine-learned model(s) can process the image data to generate an image recognition output (e.g., a recognition of the image data, a latent embedding of the image data, an encoded representation of the image data, a hash of the image data, etc.). As another example, the machine-learned model(s) can process the image data to generate an image segmentation output. As another example, the machine-learned model(s) can process the image data to generate an image classification output. As another example, the machine-learned model(s) can process the image data to generate an image data modification output (e.g., an alteration of the image data, etc.). As another example, the machine-learned model(s) can process the image data to generate an encoded image data output (e.g., an encoded and/or compressed representation of the image data, etc.). As another example, the machine-learned model(s) can process the image data to generate an upscaled image data output. As another example, the machine-learned model(s) can process the image data to generate a prediction output.

In some implementations, the input to the machine-learned model(s) of the present disclosure can be speech data. The machine-learned model(s) can process the speech data to generate an output. As an example, the machine-learned model(s) can process the speech data to generate a speech recognition output. As another example, the machine-learned model(s) can process the speech data to generate a speech translation output. As another example, the machine-learned model(s) can process the speech data to generate a latent embedding output. As another example, the machine-learned model(s) can process the speech data to generate an encoded speech output (e.g., an encoded and/or compressed representation of the speech data, etc.). As another example, the machine-learned model(s) can process the speech data to generate an upscaled speech output (e.g., speech data that is higher quality than the input speech data, etc.). As another example, the machine-learned model(s) can process the speech data to generate a textual representation output (e.g., a textual representation of the input speech data, etc.). As another example, the machine-learned model(s) can process the speech data to generate a prediction output.

In some implementations, the input to the machine-learned model(s) of the present disclosure can be latent encoding data (e.g., a latent space representation of an input, etc.). The machine-learned model(s) can process the latent encoding data to generate an output. As an example, the machine-learned model(s) can process the latent encoding data to generate a recognition output. As another example, the machine-learned model(s) can process the latent encoding data to generate a reconstruction output. As another example, the machine-learned model(s) can process the latent encoding data to generate a search output. As another example, the machine-learned model(s) can process the latent encoding data to generate a reclustering output. As another example, the machine-learned model(s) can process the latent encoding data to generate a prediction output.

In some implementations, the input to the machine-learned model(s) of the present disclosure can be statistical data. Statistical data can be, represent, or otherwise include data computed and/or calculated from some other data source. The machine-learned model(s) can process the statistical data to generate an output. As an example, the machine-learned model(s) can process the statistical data to generate a recognition output. As another example, the machine-learned model(s) can process the statistical data to generate a prediction output. As another example, the machine-learned model(s) can process the statistical data to generate a classification output. As another example, the machine-learned model(s) can process the statistical data to generate a segmentation output. As another example, the machine-learned model(s) can process the statistical data to generate a visualization output. As another example, the machine-learned model(s) can process the statistical data to generate a diagnostic output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output. As another example, the machine-learned model(s) can process the sensor data to generate a detection output.

In some cases, the machine-learned model(s) can be configured to perform a task that includes encoding input data for reliable and/or efficient transmission or storage (and/or corresponding decoding). For example, the task may be an audio compression task. The input may include audio data and the output may comprise compressed audio data. In another example, the input includes visual data (e.g., one or more images or videos), the output comprises compressed visual data, and the task is a visual data compression task. In another example, the task may comprise generating an embedding for input data (e.g., input audio or visual data).

In some cases, the input includes visual data and the task is a computer vision task. In some cases, the input includes pixel data for one or more images and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. For example, the set of categories can be foreground and background. As another example, the set of categories can be object classes. As another example, the image processing task can be depth estimation, where the image processing output defines, for each pixel in the one or more images, a respective depth value. As another example, the image processing task can be motion estimation, where the network input includes multiple images, and the image processing output defines, for each pixel of one of the input images, a motion of the scene depicted at the pixel between the images in the network input.

In some cases, the input includes audio data representing a spoken utterance and the task is a speech recognition task. The output may comprise a text output which is mapped to the spoken utterance. In some cases, the task comprises encrypting or decrypting input data. In some cases, the task comprises a microprocessor performance task, such as branch prediction or memory address translation.

FIG. 1 illustrates one example computing system that can be used to implement the present disclosure. Other computing systems can be used as well. For example, in some implementations, the user computing device 102 can include the model trainer 160 and the training dataset 162. In such implementations, the models 120 can be both trained and used locally at the user computing device 102. In some of such implementations, the user computing device 102 can implement the model trainer 160 to personalize the models 120 based on user-specific data.

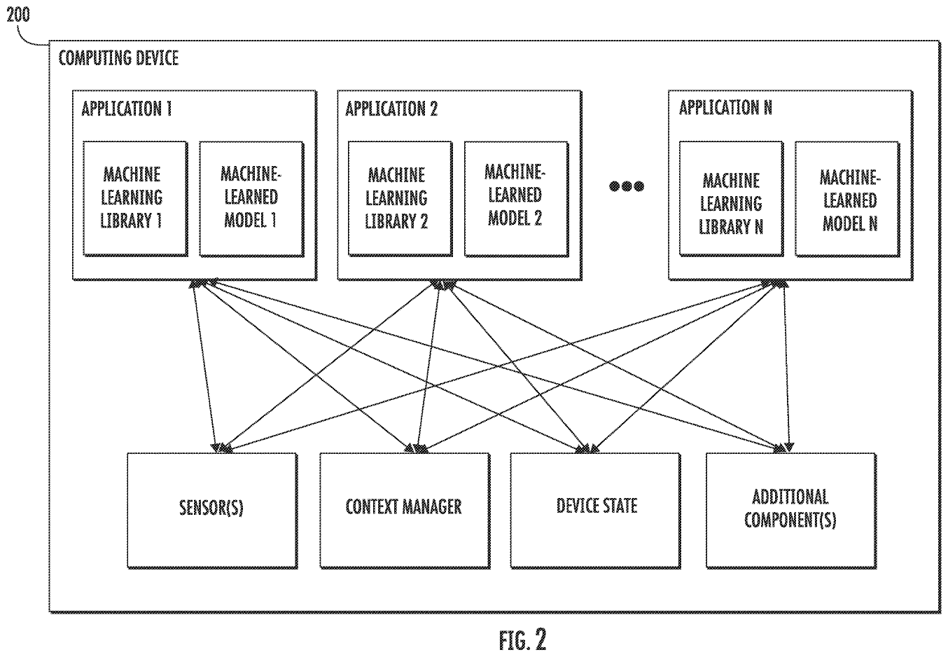

FIG. 2 depicts a block diagram of an example, non-limiting computing device 200 that can perform health-based predictions for a patient using a single time point biological sample of the patient according to example embodiments of the present disclosure. The computing device 200 can be a user computing device or a server computing device.

The computing device 200 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 2, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

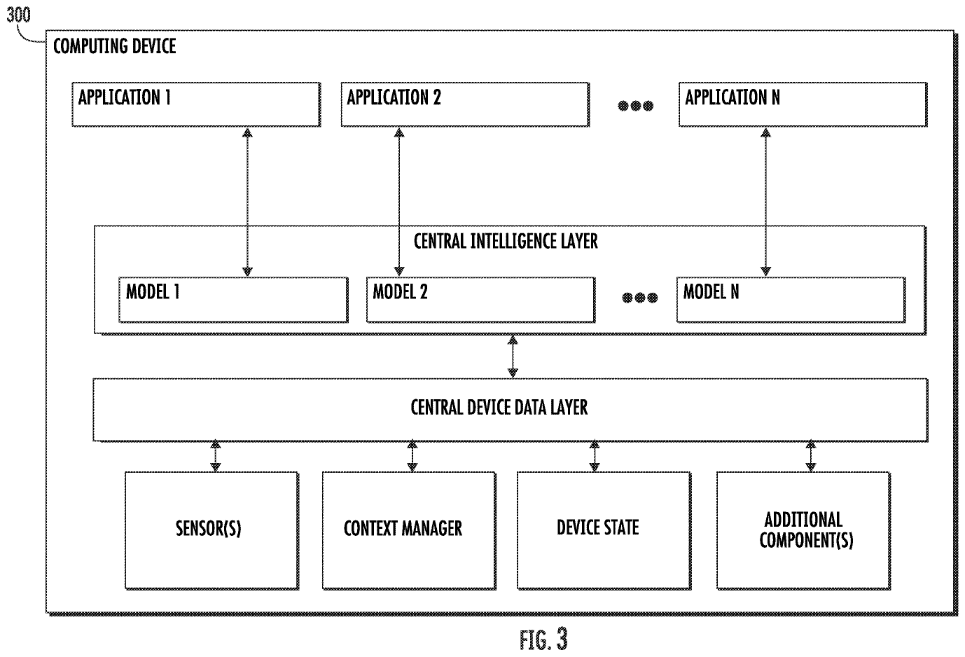

FIG. 3 depicts a block diagram of an example, non-limiting computing device 300 that can perform health-based predictions for a patient using a single time point biological sample of the patient according to example embodiments of the present disclosure. The computing device 300 can be a user computing device or a server computing device.

The computing device 300 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 3, a respective machine-learned model can be provided for each application and managed by the central intelligence layer. In other implementations, two or more applications can share a single machine-learned model. For example, in some implementations, the central intelligence layer can provide a single model for all of the applications. In some implementations, the central intelligence layer is included within or otherwise implemented by an operating system of the computing device 300.

The central intelligence layer can communicate with a central device data layer. The central device data layer can be a centralized repository of data for the computing device 300. As illustrated in FIG. 3, the central device data layer can communicate with a number of other components of the computing device, such as, for example, one or more sensors, a context manager, a device state component, and/or additional components. In some implementations, the central device data layer can communicate with each device component using an API (e.g., a private API).

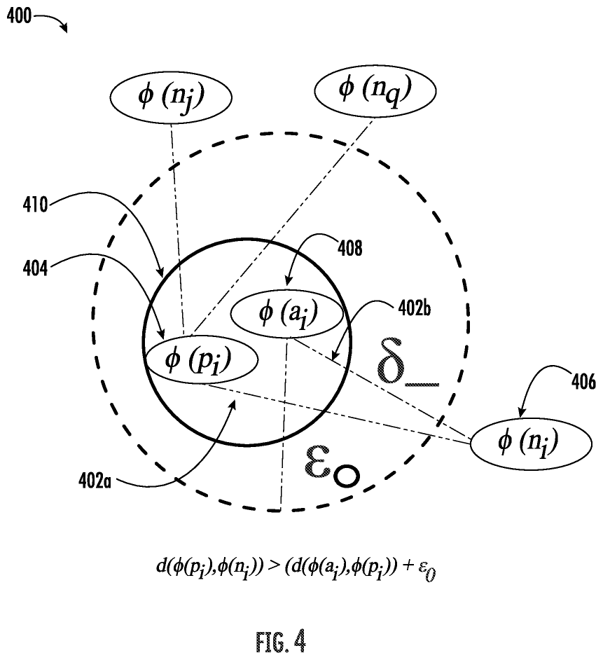

FIG. 4 depicts an example, non-limiting diagram 400 according to example embodiments of the present disclosure. Diagram 400 illustrated in the example embodiment depicted in FIG. 4 can provide illustration of how the modified triplet-based objective function of the present disclosure, and/or the regularization component that can be integrated therein, can operate (e.g., when implemented using a computing system and/or computing device described herein) to regularize distances between pairs of positive data values and negative data values in triplet tuples of a dataset with respect to anchor values in the triplet tuples. For example, in the embodiment depicted in FIG. 4, the modified triplet-based objection function and/or regularization component thereof can operate (e.g., when implemented using a computing system and/or computing device described herein) to regularize distances between pairs of positive data values and negative data values in triplet tuples of a dataset with respect to anchor values in the triplet tuples such that a first distance 402a between a positive data value 404 and a negative data value 406 in a triplet tuple of the dataset is equal to or greater than a second distance 402b between negative data value 406 and an anchor value 408 in the triplet tuple. As illustrated in the embodiment depicted in FIG. 4, a triplet-based machine learning model 120 and/or 140 that has been trained using the modified triplet-based objective function of the present disclosure can map positive pairs (e.g., positive data value 404, other positive data values, anchor value 408) inside a margin ball 410 associated with anchor data value 408.

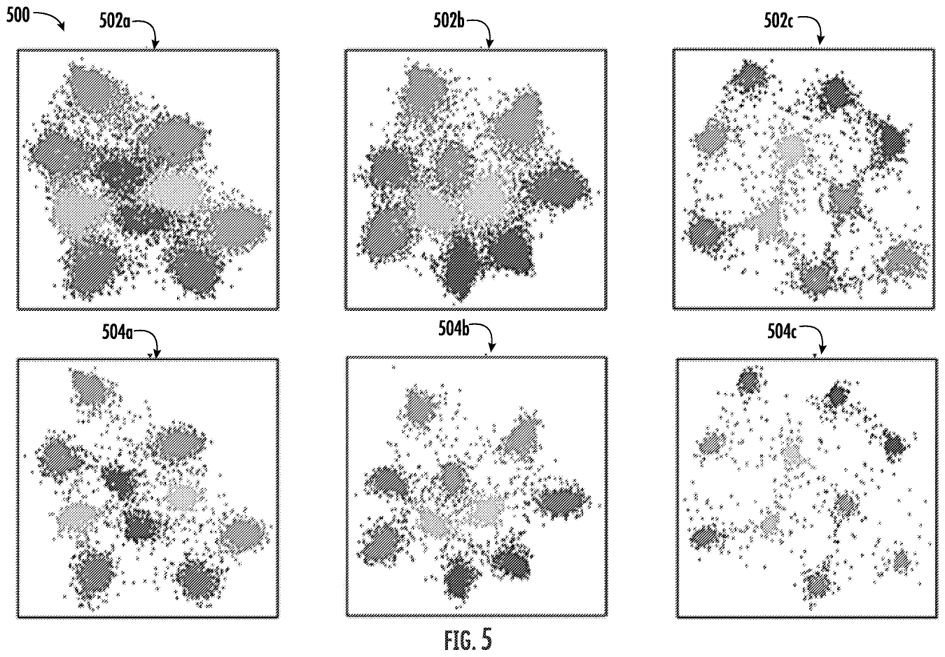

FIG. 5 depicts an example, non-limiting diagrams of training datasets 502a, 502b, 502c and testing datasets 504a, 504b, 504c according to example embodiments of the present disclosure. A triplet-based machine learning model 120 and/or 140 that has been trained using the modified triplet-based objective function of the present disclosure can learns embeddings that are relatively more compact within classes of a dataset and farther apart from other classes of the dataset with compared to existing triplet-based machine learning models, leading to better classification results as illustrated by training datasets 502a, 502b, 502c and testing datasets 504a, 504b, 504c depicted in FIG. 5.

In the example illustrated in FIG. 5, training datasets 502a, 502b correspond to implementations performed using existing triplet-based machine learning models, while training dataset 502c correspond to implementations performed using a triplet-based machine learning model 120 and/or 140 in accordance with example embodiment of the present disclosure. In the example illustrated in FIG. 5, testing datasets 504a, 504b correspond to implementations performed using existing triplet-based machine learning models, while testing dataset 504c corresponds to implementations performed using a triplet-based machine learning model 120 and/or 140 in accordance with example embodiment of the present disclosure.

### Example Methods

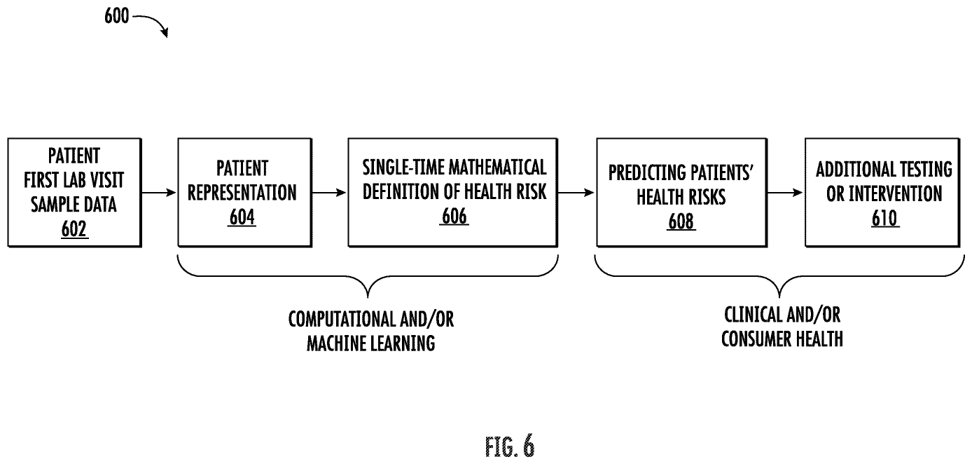

FIG. 6 illustrates a flow diagram of an example, non-limiting computer-implemented method 600 to perform health-based predictions for a patient using a single time point biological sample of the patient according to one or more example embodiments of the present disclosure. Computer-implemented method 600 may be implemented using, for instance, computing device 200, computing device 300, computing system 100, server computing system 130, and/or automated machine learning computing system 150 described above and illustrated in FIGS. 1, 2, and 3. The example embodiment illustrated in FIG. 6 depicts operations performed in a particular order for purposes of illustration and discussion. Those of ordinary skill in the art, using the disclosures provided herein, will understand that various operations or steps of computer-implemented method 600 or any of the other methods disclosed herein may be adapted, modified, rearranged, performed simultaneously, include operations not illustrated, and/or altered in various ways without deviating from the scope of the present disclosure.

At 602, a computing system can obtain (e.g., receive) patient lab visit sample data 602 (e.g., a single time point biological sample of the patient). At 604 and 606, the computing system can perform one or more computational and/or machine learning operations (e.g., patient representation 604 and single-time mathematical definition of health risk 606) in accordance with one or more embodiments to facilitate one or more clinical and/or consumer health operations such as, for instance: predicting patients' health risks 608 using, for instance, one or more triplet-based machine learning models 120 and/or 140; and/or additional testing or intervention 610.

FIG. 7 illustrates a flow diagram of an example, non-limiting computer-implemented method 700 to perform health-based predictions for a patient using a single time point biological sample of the patient according to one or more example embodiments of the present disclosure. Computer-implemented method 700 may be implemented using, for instance, computing device 200, computing device 300, computing system 100, server computing system 130, and/or automated machine learning computing system 150 described above and illustrated in FIGS. 1, 2, and 3. The example embodiment illustrated in FIG. 7 depicts operations performed in a particular order for purposes of illustration and discussion. Those of ordinary skill in the art, using the disclosures provided herein, will understand that various operations or steps of computer-implemented method 700 or any of the other methods disclosed herein may be adapted, modified, rearranged, performed simultaneously, include operations not illustrated, and/or altered in various ways without deviating from the scope of the present disclosure.

At 702, a computer-implemented method 700 can include integrating, by a computing system (e.g., computing system 100, server computing system 130, automated machine learning computing system 150) comprising one or more computing devices (e.g., computing device 200, computing device 300, computing device 102), a regularization component into a triplet-based objective function associated with a triplet-based machine learning model to create a modified triplet-based objective function. In some embodiments, the regularization component can operate (e.g., when implemented by computing system 100) to regularize distances (e.g., distances 402a, 402b) between pairs of positive data values (e.g., positive data value 404) and negative data values (e.g., negative data value 406) in triplet tuples of a dataset (e.g., a healthcare dataset) with respect to anchor values (e.g., anchor value 408) in the triplet tuples. In these or other embodiments, the dataset can include a plurality of data values represented graphically in a multi-dimensional space (e.g., a Euclidean space).

For example, the dataset can constitute and/or include a healthcare dataset that can include genetic features, phenotypic features, and/or lifestyle features.

At 704, a computer-implemented method 700 can include training, by the computing system (e.g., by computing system 100 and/or automated machine learning computing system 150 using model trainer 161), a triplet-based machine learning model using the dataset and the modified triplet-based objective function to create a trained triplet-based machine learning model (e.g., a trained triplet-based machine learning model 120 and/or 140).

At 706, a computer-implemented method 700 can include generating, by the computing system, one or more health-based predictions for a patient using the trained triplet-based machine learning model and a single time point biological sample of the patient.

### Additional Disclosure

As referenced herein, the term “entity” refers to a human, a user, an end-user, a consumer, a computing device and/or program (e.g., a processor, computing hardware and/or software, an application, etc.), an agent, a machine learning (ML) and/or artificial intelligence (AI) algorithm, model, system, and/or application, and/or another type of entity that can implement and/or facilitate implementation of one or more embodiments of the present disclosure as described herein, illustrated in the accompanying drawings, and/or included in the appended claims. As referred to herein, the terms “includes” and “including” are intended to be inclusive in a manner similar to the term “comprising.” As referenced herein, the terms “or” and “and/or” are generally intended to be inclusive, that is (i.e.), “A or B” or “A and/or B” are each intended to mean “A or B or both.” As referred to herein, the terms “first,” “second,” “third,” and so on, can be used interchangeably to distinguish one component or entity from another and are not intended to signify location, functionality, or importance of the individual components or entities. As referenced herein, the terms “couple,” “couples,” “coupled,” and/or “coupling” refer to chemical coupling (e.g., chemical bonding), communicative coupling, electrical and/or electromagnetic coupling (e.g., capacitive coupling, inductive coupling, direct and/or connected coupling, etcetera (etc.)), mechanical coupling, operative coupling, optical coupling, and/or physical coupling.

Numerous details are set forth in the foregoing description. However, it will be apparent to one of ordinary skill in the art having the benefit of this disclosure that the present disclosure may be practiced without these specific details. In some instances, structures and devices are shown in block diagram form, rather than in detail, to avoid obscuring the present disclosure.

Some portions of the detailed description have been presented in terms of processes and symbolic representations of operations on data bits within a computer memory. Here, a process can include a self-consistent sequence of steps leading to a result. The steps can include those requiring physical manipulations of physical quantities. These quantities can take the form of electrical or magnetic signals capable of being stored, transferred, combined, compared, and otherwise manipulated. These signals can be referred to as bits, values, symbols, characters, terms, numbers, or the like. These terms and similar terms can be associated with physical quantities and can represent labels applied to these quantities. The terms including “analyzing,” “accessing,” “determining,” “identifying,” “adjusting,” “modifying,” “transmitting,” “receiving,” “processing,” “generating,” or the like, can refer to the actions and processes of a computer system, a computing device, or similar electronic computing device, that manipulates and transforms data represented as physical (e.g., electronic) quantities within the computer system's registers and memories into other data that can be similarly represented as physical quantities within the computer system's memories, registers, or other information storage device, data transmission device, or data processing device.

Certain examples of the present disclosure can relate to an apparatus that can be implemented to perform the operations described herein. This apparatus can include a computing device that can be activated or reconfigured by a computer program comprising electronic instructions stored in the computing device. Such a computer program may be stored in a computer-readable storage medium (e.g., non-transitory computer-readable storage medium), which can include any type of storage. For example, the storage can include hard disk drives, solid state drives, floppy disks, optical disks, read-only memories (ROMs), compact disc read-only memories (CD-ROMs), magnetic-optical disks, random access memories (RAMs), programmable ROM (PROM), erasable PROMs (EPROMs), electrically erasable PROMs (EEPROMs), magnetic or optical cards, or any type of media suitable for storing electronic instructions.

The technology discussed herein makes reference to servers, databases, software applications, and other computer-based systems, as well as actions performed by, and information sent to and from such systems. The inherent flexibility of computer-based systems allows for a wide variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure covers such alterations, variations, and equivalents.

The above description is intended to be illustrative, and not restrictive. The scope of the disclosure can therefore be determined with reference to the claims.

1. A computing system, the computing system comprising:
one or more processors; and
one or more non-transitory computer-readable media that collectively store instructions that, when executed by the one or more processors, cause the computing system to perform operations, the operations comprising:
training a triplet-based machine learning model using a dataset and a modified triplet-based objective function to create a trained triplet-based machine learning model, the dataset comprising a healthcare dataset having at least one of genetic features, phenotypic features, or lifestyle features;
receiving a single time point biological sample of a patient; and
generating one or more health-based predictions for the patient using the trained triplet-based machine learning model and the single time point biological sample of the patient,
wherein the modified triplet-based objective function comprises a regularization component that operates to regularize distances between pairs of positive data values and negative data values in triplet tuples of the dataset with respect to anchor values in the triplet tuples.

2. The computing system of claim 1, the regularization component operable to regularize the distances between the pairs of the positive data values and the negative data values in the triplet tuples of the dataset with respect to the anchor values in the triplet tuples such that a first distance between a positive data value and a negative data value in a triplet tuple is equal to or greater than a second distance between the negative data value and an anchor value in the triplet tuple.

3. The computing system of claim 1, the triplet-based machine learning model comprising a triplet-based deep metric learning model, and the trained triplet-based machine learning model comprising a trained triplet-based deep metric learning model.

4. The computing system of claim 1, the multi-dimensional space comprising a Euclidean space, and the first distance, the second distance, and each of the distances between the pairs of the positive data values and the negative data values in the triplet tuples comprising a Euclidean distance.

5. The computing system of claim 1, the modified triplet-based objective function operable to learn similarity-based embeddings of the plurality of data values in the dataset during training of the triplet-based machine learning model using the dataset and the modified triplet-based objective function.

6. The computing system of claim 1, the modified triplet-based objective function operable to provide improved density of embeddings of data values of each class of data values in the dataset to provide improved classification results across different classifier models that implement the modified triplet-based objective function compared to classifier models that implement a different objective function.

7. The computing system of claim 1, the modified triplet-based objective function operable to provide improved separability between different classes of data values in the dataset to provide improved classification results across different classifier models that implement the modified triplet-based objective function compared to classifier models that implement a different objective function.

8. The computing system of claim 1, the modified triplet-based objective function operable to provide improved separability between first embeddings of first data values of a first class of data values in the dataset and second embeddings of second data values of a second class of data values in the dataset to provide improved classification results across different classifier models that implement the modified triplet-based objective function compared to classifier models that implement another objective function.

9. The computing system of claim 1, the one or more health-based predictions comprising at least one of a classification of the patient in one or more predefined health-based classes, a health risk score prediction, a diagnosis prediction, or a prognosis prediction.

10. A computer-implemented method, the computer-implemented method comprising:
integrating, by a computing system comprising one or more computing devices, a regularization component into a triplet-based objective function associated with a triplet-based machine learning model to create a modified triplet-based objective function, the regularization component operable to regularize distances between pairs of positive data values and negative data values in triplet tuples of a dataset with respect to anchor values in the triplet tuples, the dataset comprising a healthcare dataset having at least one of genetic features, phenotypic features, or lifestyle features;
training, by the computing system, a triplet-based machine learning model using the dataset and the modified triplet-based objective function to create a trained triplet-based machine learning model;
receiving a single time point biological sample of a patient; and
generating, by the computing system, one or more health-based predictions for the patient using the trained triplet-based machine learning model and the single time point biological sample of the patient.

11. The computer-implemented method of claim 10, the regularization component operable to regularize the distances between the pairs of the positive data values and the negative data values in the triplet tuples of the dataset with respect to the anchor values in the triplet tuples such that a first distance between a positive data value and a negative data value in a triplet tuple of the dataset is equal to or greater than a second distance between the negative data value and an anchor value in the triplet tuple.

12. The computer-implemented method of claim 10, the triplet-based machine learning model comprising a triplet-based deep metric learning model, and the trained triplet-based machine learning model comprising a trained triplet-based deep metric learning model.

13. The computer-implemented method of claim 10, the modified triplet-based objective function operable to learn similarity-based embeddings of the plurality of data values in the dataset during training, by the computing system, of the triplet-based machine learning model using the dataset and the modified triplet-based objective function.

14. The computer-implemented method of claim 10, the one or more health-based predictions comprising at least one of a classification of the patient in one or more predefined health-based classes, a health risk score prediction, a diagnosis prediction, or a prognosis prediction.

15. A computing device, the computing device comprising:
one or more processors; and
one or more non-transitory computer-readable media that collectively store instructions that, when executed by the one or more processors, cause the computing device to perform operations, the operations comprising:
integrating a regularization component into a triplet-based objective function associated with a triplet-based machine learning model to create a modified triplet-based objective function, the regularization component operable to regularize distances between pairs of positive data values and negative data values in triplet tuples of a dataset with respect to anchor values in the triplet tuples, the dataset comprising a healthcare dataset having at least one of genetic features, phenotypic features, or lifestyle features; and
training a triplet-based machine learning model using the dataset and the modified triplet-based objective function to create a trained triplet-based machine learning model that, when implemented, generates one or more health-based predictions for a patient based on a single time point biological sample of the patient.

16. The computing device of claim 15, the regularization component operable to regularize the distances between the pairs of the positive data values and the negative data values in the triplet tuples of the dataset with respect to the anchor values in the triplet tuples such that a first distance between a positive data value and a negative data value in a triplet tuple of the dataset is equal to or greater than a second distance between the negative data value and an anchor value in the triplet tuple.

17. The computing device of claim 15, the triplet-based machine learning model comprising a triplet-based deep metric learning model, and the trained triplet-based machine learning model comprising a trained triplet-based deep metric learning model.

18. The computing device of claim 15, the modified triplet-based objective function operable to learn similarity-based embeddings of the plurality of data values in the dataset during training, by the computing system, of the triplet-based machine learning model using the dataset and the modified triplet-based objective function.

19. The computing device of claim 15, the one or more health-based predictions comprising at least one of a classification of the patient in one or more predefined health-based classes, a health risk score prediction, a diagnosis prediction, or a prognosis prediction.
